(19) United States
(12) Patent Application Publication
(10) Pub. No.: US 2022/0040179 A1
(43) Pub. Date: Feb. 10, 2022

(54) USE OF CDK8/19 INHIBITORS FOR TREATMENT OF ESTABLISHED COLON CANCER HEPATIC METASTASIS

(71) Applicant: University of South Carolina, Columbia, SC (US)

(72) Inventors: Jaixin LIANG, Columbia, SC (US); Igor B. RONINSON, Lexington, SC (US)

(21) Appl. No.: 16/488,381

(22) PCT Filed: Feb. 23, 2018
(86) PCT No.: PCT/US2018/019362

§ 371 Date: Aug. 23, 2019

Related U.S. Application Data
(60) Provisional application No. 62/462,528, filed on Feb. 23, 2017.

(51) Int. Cl. A61K 31/517; A61P 35/04

(52) U.S. Cl. CPC A61K 31/517; A61P 35/04

(57) ABSTRACT

The invention relates to the treatment of cancer. More particularly, the invention relates to the treatment of metastatic cancer. The invention provides new treatments for colon cancer patients who develop metastasis in the liver. The invention provides a method for treating hepatic metastatic colon cancer in a subject, the method comprising administering to the subject a small molecule selective inhibitor of CDK8/19 at a dosage that inhibits growth of the hepatic metastatic colon cancer, and does not cause a dose-limiting toxicity. The invention further provides a method for treating a subject having both a primary colon cancer tumor and hepatic metastatic colon cancer, the method comprising administering to the subject a small molecule selective inhibitor of CDK8/19 at a dosage that inhibits growth of the hepatic metastatic colon cancer, but does not significantly inhibit growth of the primary colon cancer tumor.

### REFERENCE TO THE SEQUENCE LISTING SUBMITTED VIA EFS-WEB

This application contains a sequence listing submitted via EFS-Web. The content of the ASCII text file of the sequence listing named “169958_00010_ST25.txt” which is 1.65 kb in size was created on Jun. 23, 2020 and electronically submitted via EFS-Web. The sequence listing is incorporated herein by reference in its entirety.

### BACKGROUND OF THE INVENTION

### Field of the Invention

The invention relates to the treatment of cancer. More particularly, the invention relates to the treatment of metastatic cancer.

### Summary of the Related Art

Cyclin-dependent kinase 8 (CDK8) and its paralog CDK19 are two closely related (80% identity) serine/threonine kinases (Galbraith et al., 2010; Tsutsui et al., 2011) that, unlike better-known CDK (cyclin-dependent kinase) family members, such as CDK1 (CDCl₂), CDK2 or CDK4/6, do not play a general role in cell cycle progression. CDK8 depletion does not inhibit the growth of normal cells (Westerling et al., 2007), global Cre/Lox-mediated CDK8 knockout in adult mouse tissues has no phenotypic consequences (McCleland et al., 2015) and small molecule CDK8/19 inhibitors do not generally suppress cell proliferation (Porter et al., 2012). A key function of CDK8/19 is phosphorylation of the C-terminal domain (CTD) of RNA polymerase II (Pol II), enabling the elongation of transcription; CDK8/19 exert this activity not globally but only in the context of genes that become activated by transcription-inducing factors (Donner et al., 2010; Galbraith et al., 2013). Consequently, CDK8/19 inhibition has little effect under homeostatic conditions, but it prevents transcriptional reprogramming triggered by various signals (Donner et al., 2010; Galbraith et al., 2013).

CDK8/19-mediated transcriptional reprogramming is especially pertinent in cancer, where CDK8 has been identified as a transcriptional regulator in several signaling pathways implicated in carcinogenesis and metastasis, including Wnt/-catenin (Firestein et al., 2008), Notch (Fryer et al., 2004), the serum response network (Donner et al., 2010), TGF (Alarcon et al., 2009), HIF1A (Galbraith et al., 2013) and NFKB (US20140309224A1). CDK8 has been identified as an oncogene, capable of transforming NIH-3T3 cells and amplified in colorectal cancers (Firestein et al., 2008), implicated in breast cancer (Broude et al., 2015; McDermott et al., 2017; Porter et al., 2012; Xu et al., 2015a), melanoma (Kapoor et al., 2010) and pancreatic cancer (Xu et al., 2015b) and associated with the cancer stem cell phenotype (Adler et al., 2012). CDK8 depletion was also found to increase tumor surveillance activity of natural killer (NK) cells (Putz et al., 2013). Our work has identified CDK8/19 as a mediator of damage-induced gene expression associated with tumor-promoting paracrine activities, invasion and metastasis (Porter et al., 2012). CDK8/19 inhibition was also shown to decrease the expression of genes associated with invasion and metastasis in prostate cancer (Bragelmann et al., 2016). Hence, CDK8/19 provides an attractive anticancer drug target. Many different groups are developing small-molecule CDK8/19 inhibitors (Rzymski et al., 2015). Some examples of such inhibitors include marine alkaloid Cortistatin A (CsA) and its derivatives (Cee et al., 2009; Pelish et al., 2015) (WO2015100420A1), Senexin A (Porter et al., 2012) (U.S. Pat. No. 8,598,344), Senexin B (U.S. Pat. No. 9,409,873), SEL120-34A (Zylkiewicz et al., 2016), compounds 13 and 32 (Koehler et al., 2016), CCT251921 (Mallinger et al., 2016) and MSC2530818 (Czodrowski et al., 2016).

CDK8 was originally identified as an oncogene in colon cancer and CDK8 knockdown was reported to inhibit colon cancer cell growth (Firestein et al., 2008). However, studies by several groups including ours failed to detect significant growth inhibition in colon cancer cells, including those that overexpress CDK8, when the cells were treated with CDK8/19 kinase inhibitors (Koehler et al., 2016; Pelish et al., 2015; Porter et al., 2012). Treatment of colon cancer liver metastases is an unmet medical need, which applies to approximately 14.5% of all colon cancer patients, who develop metastasis in the liver (Manfredi et al., 2006).

We have previously reported (in poster presentations) that, in a spleen-to-liver metastasis model of syngeneic mouse CT26 colon cancer, both Senexin B treatment of mice and CDK8 knockdown in tumor cells suppressed metastatic growth in the liver without a significant effect on primary tumor growth in the spleen (Porter et al., 2014, 2015). Our results presented in those posters (corresponding to FIGS. 4 and 5 in the present application) indicated that CDK8/19 inhibitors could prevent colon cancer liver metastasis but not that such inhibitors could be used for treatment of already established metastases.

There is, therefore, a need for new treatments for colon cancer patients who develop metastasis in the liver.

### BRIEF SUMMARY OF THE INVENTION

The invention relates to the treatment of cancer. More particularly, the invention relates to the treatment of metastatic cancer. The invention provides new treatments for colon cancer patients who develop metastasis in the liver. In a previous study, authors concluded that suppression of a primary colon cancer xenograft growth in vivo was achieved by using high doses of CDK8/19 inhibitors that also induced pronounced toxicity (Clarke et al., 2016). We have now discovered that CDK8/19 inhibition using lower, non-toxic dosages of CDK8/19 inhibitors suppresses the growth of colon cancer hepatic metastases once such metastases have already been established. This discovery was surprising, given the lack of efficacy of CDK8/19 inhibitors against primary colon cancers. Our findings indicate that CDK8/19 inhibitors can be safely used for the treatment of colon cancer metastatic growth in the liver, even when such inhibitors have little or no effect on the primary tumor growth.

### BRIEF DESCRIPTION OF THE DRAWINGS

FIG. 1A shows the results of quantitative reverse-transcription PCR (QPCR) assays for CDK8 and CDK19 in CT26 cells, carried out as previously described (McDermott et al., 2017) using the following pairs of PCR primers: CGGGTCGAGGACCTGTTTG (SEQ ID NO:1) and TGCCGACATAGAAATTCCAGTTC (SEQ ID NO:2) for CDK8; and GGTCAAGCCTGACAGCAAAGT (SEQ ID NO:3) and TTCCTGGAAGTAAGGGTCCTG (SEQ ID NO:4) for CDK19. FIG. 1B shows results of QPCR assays for CDK8 mRNA in CT26 cells into which were delivered two shRNAs targeting sequences, CTAACGTCAGAACCAATATTT (shCDK8-1; SEQ ID NO:5) and GTCTTATCAGTGGGTTGATTC (shCDK8-2; SEQ ID NO:6), using the pLK0.1 lentiviral vector, as previously described (Porter et al., 2012). FIG. 1C shows results of immunoblotting assays for CDK8 protein carried out using goat anti-CDK8 antibody (Santa Cruz, sc-1S21) as previously described (Porter et al., 2012) in the wild-type CT26 cells and in cells transduced with shCDK8-1 and shCDK8-2.

FIG. 2A shows that both shRNAs inhibited CT26 cell growth. FIG. 2B shows that Senexin B (1 uM) produced no significant growth inhibition in a S-day assay.

FIG. 3A shows the dynamics of tumor growth and final tumor weights for CT26 cells transduced with insert-free pLK0.1 (vector control) or shCDK8-1. FIG. 3B compares s.c. tumor growth of unmodified CT26 cells in mice treated with Senexin B dimaleate.

FIG. 4A shows spleen photographs revealing extensive tumor growth in the spleen, as well as spleen weights in two groups of mice in a splenic injection model, where tumor cells are injected in the spleen, from where they metastasize into the liver (Senexin B treatment v. vehicle control). FIG. 4B shows liver photographs revealing tumor growth, liver weights in the two groups, and image quantitation of the tumor area in the liver.

FIG. 5A shows that, in a similar study with vector control and shCDK8-1 CT26 cells (no treatment), the average spleen weight was decreased in shCDK8-1 cells relative to the control, but the difference didn't reach statistical significance. FIG. SB shows that, in contrast, the liver weight (reflecting metastatic growth) was significantly inhibited upon CDK8 knockdown.

FIG. 6A shows macroscopically and microscopically detectable metastatic tumors in the livers of mice sacrificed 7 days after splenic tumor cell inoculation. FIG. 6B shows the weights of livers collected two weeks after tumor cell inoculation, when Senexin B was administered two days prior to splenic inoculation, during the first week after inoculation, during the second week after inoculation, or over the entire

FIG. 7A shows extensive liver metastasis in sacrificed BALB/c mice after splenic injection of 2×10⁵ CT26 cells into the mice, followed by removal of the spleen 1 minute after injection. FIG. 7B shows that mice injected with shCDK8-1 cells showed significantly extended survival relative to mice inoculated with vector control cells, that Senexin B dimaleate treatment prolonged the survival of mice inoculated with vector control cells to a similar degree as the survival time of mice inoculated with shCDK8-1 cells, and that the combination of knockdown of CDK8 and treatment with Senexin B together increased the survival time even further. FIG. 7C shows KM survival plots for mice receiving control diet (Research Diets, D12450B), low-dose Senexin B diet and high-dose Senexin B diet, along with the measurements of Senexin B serum concentrations in mice receiving the low- and the high-dose diets.

FIG. 8A shows weights of livers with tumor metastases after splenic injection in athymic nude mice of human HCTl 16 cells. FIG. 8B shows liver weights demonstrating strong inhibition of hepatic tumor growth by Senexin B in this colon cancer cell linetwo-week period following splenic inoculation of CT26 cells.

### DETAILED DESCRIPTION OF THE PREFERRED EMBODIMENTS

The invention relates to the treatment of cancer. More particularly, the invention relates to the treatment of metastatic cancer. The invention provides new treatments for colon cancer patients who develop metastasis in the liver.

In a previous study, authors concluded that suppression of a primary colon cancer xenograft growth in vivo was achieved by using high doses of CDK8/19 inhibitors that also induced pronounced toxicity (Clarke et al., 2016). We have now discovered that CDK8/19 inhibition using lower, non-toxic dosages of CDK8/19 inhibitors suppresses the growth of colon cancer hepatic metastases once such metastases have already been established. This discovery was surprising, given the lack of efficacy of CDK8/19 inhibitors against primary colon cancers. Our findings indicate that CDK8/19 inhibitors can be safely used for the treatment of colon cancer metastatic growth in the liver, even when such inhibitors have little or no effect on the primary tumor growth.

In a first aspect, the invention provides a method for treating hepatic metastatic colon cancer in a subject, the method comprising administering to the subject a small molecule selective inhibitor of CDK8/19 at a dosage that inhibits growth of the hepatic metastatic colon cancer, and does not cause a dose-limiting toxicity.

In a second aspect, the invention provides a method for treating a subject having both a primary colon cancer tumor and hepatic metastatic colon cancer, the method comprising administering to the subject a small molecule selective inhibitor of CDK8/19 at a dosage that inhibits growth of the hepatic metastatic colon cancer, but does not significantly inhibit growth of the primary colon cancer tumor. In some embodiments, this aspect of the invention further comprises treating the primary colon cancer tumor. In some embodiments, treatment of the primary colon cancer tumor comprises surgery. In some embodiments, treatment of the primary colon cancer tumor comprises radiation therapy. In some embodiments, treatment of the primary colon cancer tumor comprises chemotherapy.

For purposes of the invention, a “small molecule selective inhibitor of CDK8/19” is a small molecule compound that inhibits one or more of CDK8 and CDK19 to a greater extent than it inhibits certain other CDKs. In some embodiments, such compounds further inhibit CDK8/19 to a greater extent than CDK9. In preferred embodiments, such greater extent is at least 2-fold more than CDK9. A “small molecule compound” is a molecule having a formula weight of about 800 Daltons or less. For purposes of the invention, a “dose-limiting toxicity” is a toxicity associated with a dosage of the small molecule selective inhibitor of CDK8/19 sufficient to proscribe the further administration of the small molecule selective inhibitor of CDK8/19 at such a dosage in an FDA-approved clinical trial.

Many small molecule selective inhibitors of CDK8/19 are known, and additional ones are continuing to be discovered, including, without limitation, marine alkaloid Cortistatin A (CsA) and its derivatives (Cee et al., 2009; Pelish et al., 2015) (WO2015100420A1), Senexin A (Porter et al., 2012) (U.S. Pat. No. 8,598,344), Senexin B (U.S. Pat. No. 9,409,873), SEL120-34A (Zylkiewicz et al., 2016), compounds 13 and 32 (Koehler et al., 2016), CCT251921 (Mallinger et al., 2016) and MSC2530818 (Czodrowski et al., 2016), each of which are hereby incorporated by reference in their entirety. These, as well as newly discovered small molecule selective inhibitors of CDK8/19 are within the scope of the invention.

The following examples are intended to further illustrate certain preferred embodiments of the invention, and are not to be construed as limiting the scope of the invention.

### Example 1. Treatment with CDKS/19 Inhibitor or shRNA Knockdown of CDKS in CT26 Colon Cancer Cells Suppresses Metastatic Growth in the Liver

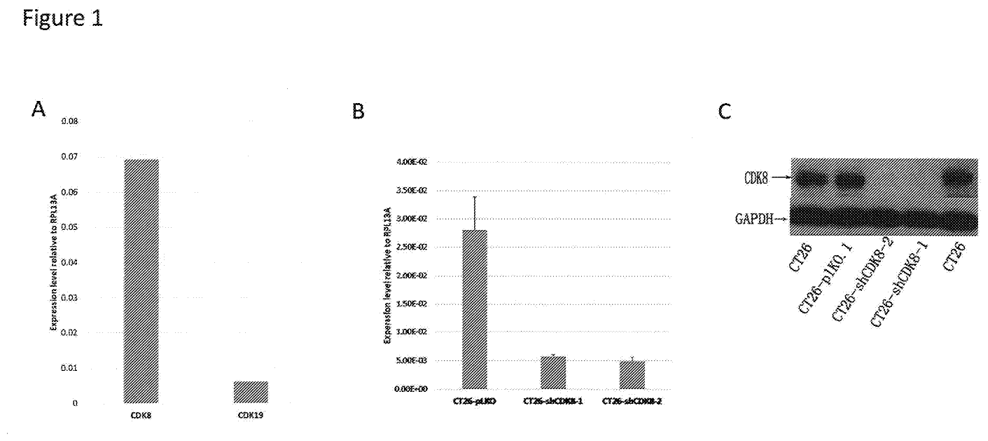

To investigate the role of CDK8/19 in colon cancer growth and metastasis, we used murine CT26 colon cancer cell line, derived from a BALB/c mouse following chemical carcinogenesis (Griswold and Corbett, 1975). Cells were propagated in RPMI1640 medium with 10% Fetal Bovine Serum. FIG. 1A shows the results of quantitative reverse-transcription PCR (QPCR) assays for CDK8 and CDK19 in CT26 cells, carried out as described (McDermott et al., 2017) using the following pairs of PCR primers: CGGGTCGAGGACCTGTTTG (SEQ ID NO:1) and TGCCGACATAGAAATTCCAGTTC (SEQ ID NO:2) for CDK8 and GGTCAAGCCTGACAGCAAAGT (SEQ ID NO:3) and

TTCCTGGAAGTAAGGGTCCTG (SEQ ID NO:4) for CDK19. The results in FIG. 1A demonstrate that the expression of CDK19 in CT26 cells is very low relative to CDK8, and therefore only CDK8-targeting shRNAs needed to be used for stable knockdown of CDK8/19 in this cell line. Two shRNAs targeting sequences CTAACGTCAGAACCAATATTT (shCDK8-1; SEQ ID NO:5) and GTCTTATCAGTGGGTTGATTC (shCDK8-2; SEQ ID NO:6) in murine CDK8 mRNA were delivered into CT26 cells using pLK0.1 lentiviral vector, as described (Porter et al., 2012). FIG. 1B shows QPCR assays for CDK8 mRNA and FIG. 1C shows immunoblotting assays for CDK8 protein carried out using goat anti-CDK8 antibody (Santa Cruz, sc-1521) as described (Porter et al., 2012) in the wild-type CT26 cells and in cells transduced with shCDK8-1 and shCDK8-2. The results in FIG. 1B and FIG. 1C indicate efficient CDK8 knockdown with both shRNAs.

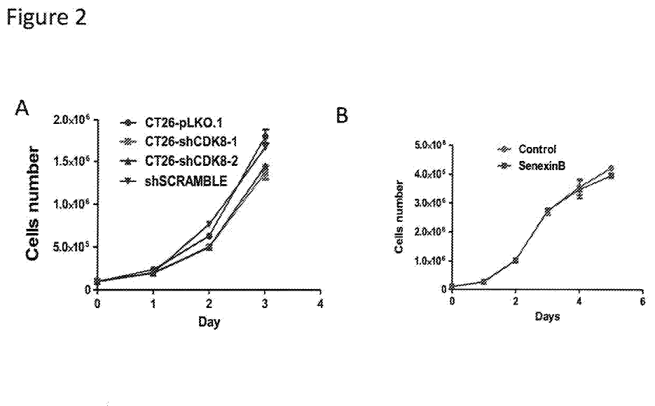

We have tested the effects of CDK8/19 inhibition on in vitro growth of CT26 cells using shCDK8-1 and shCDK8-2 or selective small-molecule CDK8/19 kinase inhibitor Senexin B. Both shRNAs inhibited CT26 cell growth (FIG. 2A) but Senexin B (1 uM) produced no significant growth inhibition in a 5-day assay (FIG. 2B). These results are consistent with the disparity between the effects of CDK8 shRNA and small-molecule CDK8/19 inhibitors on in vitro growth of human HCT116 colon cancer cells that overexpress CDK8 (Firestein et al., 2008; Koehler et al., 2016; Pelish et al., 2015; Porter et al., 2012).

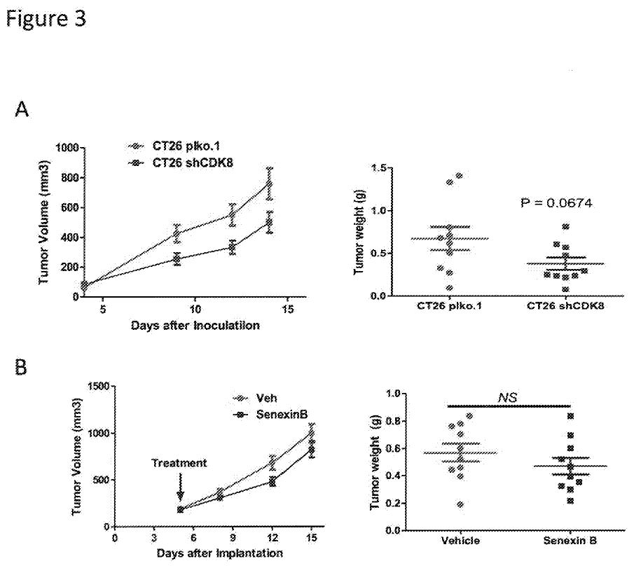

We further investigated the effects of CDK8/19 inhibition or knockdown on the growth of primary CT26 tumors implanted subcutaneously (s.c.), at I xl 0⁶ cells, in 8 weeks-old female BALB/c mice (n=I0). FIG. 3A compares the dynamics of tumor growth and final tumor weights for CT26 cells transduced with insert-free pLK0.1 (vector control) or shCDK8-1. CDK8 shRNA appeared to inhibit tumor growth s.c. but its effect did not reach statistical significance. FIG. 3B compares s.c. tumor growth of unmodified CT26 cells in mice treated with Senexin B dimaleate, administered by gavage at 50 mg/kg doses (weight doses calculated for Senexin B dimaleate rather than free Senexin B) in 6.25% 2-Hydroxypropyl-cyclodextrin, I % Dextrose buffer (CD vehicle), b.i.d. or with the vehicle control. The effect of Senexin B treatment on s.c. tumor growth was not statistically significant.

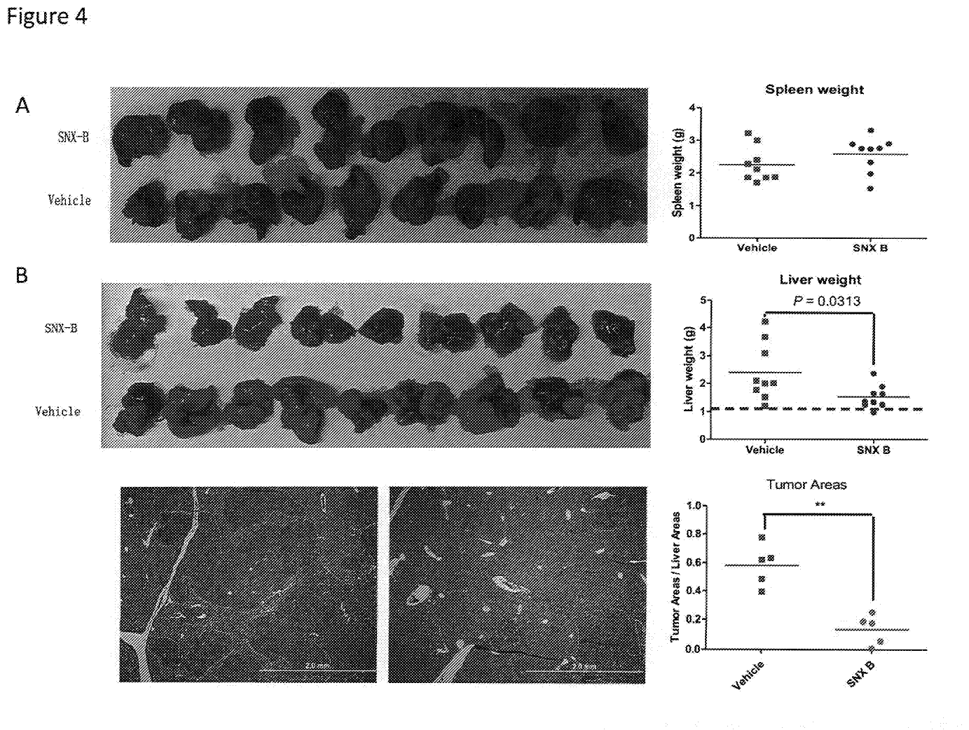

To compare the effects of CDK8/19 inhibition on primary and metastatic tumor growth, we used a splenic injection model (Lafreniere and Rosenberg, 1986; Zhang et al., 2009), where tumor cells are injected in the spleen, from where they metastasize into the liver. In the study shown in FIG. 4, 2×10⁵ CT26 cells were injected into the spleens of 8 weeks old female BALB/c mice, and mice were treated by daily i.p. injection of Senexin B dichloride (40 mg/kg) in 10 mM citrate buffer, pH6, 150 mM NaCl or with vehicle control (n=9). 1 6 days later, mice were sacrificed and the weights of tumor-containing spleens (primary tumor) and livers (metastatic tumor) were measured. FIG. 4A shows spleen photographs revealing extensive tumor growth in the spleen, as well as spleen weights in the two groups. Senexin B treatment had no effect on the primary tumor growth in the spleen. FIG. 4B shows liver photographs revealing tumor growth, liver weights in the two groups, and image quantitation of the tumor area in the liver (generated microscopically after H&E staining of sections of 5 tumors in each group). The results show that Senexin B strongly inhibited metastatic tumor growth in the liver.

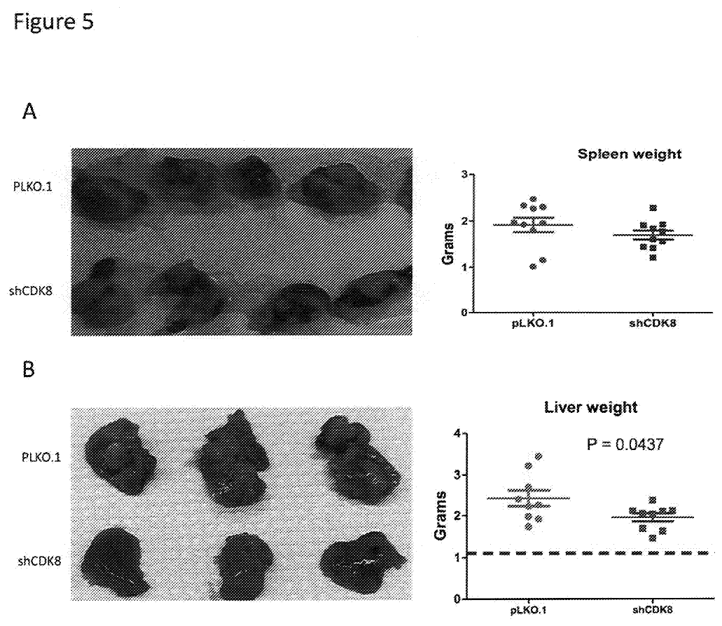

FIG. 5 shows the results of a similar study with vector control and shCDK8-1 CT26 cells (no treatment). Mice were sacrificed 16 days after tumor cell inoculation into the spleen and spleens and livers were weighed. Although the average spleen weight was decreased in shCDK8-1 cells relative to the control (FIG. 5A), the difference didn't reach statistical significance. In contrast, the liver weight (reflecting metastatic growth) was significantly inhibited upon CDK8 knockdown (FIG. 5B), in agreement with the results obtained with Senexin B.

### Example 2. Senexin B Treatment Suppresses the Growth of Already-Established Liver Metastases

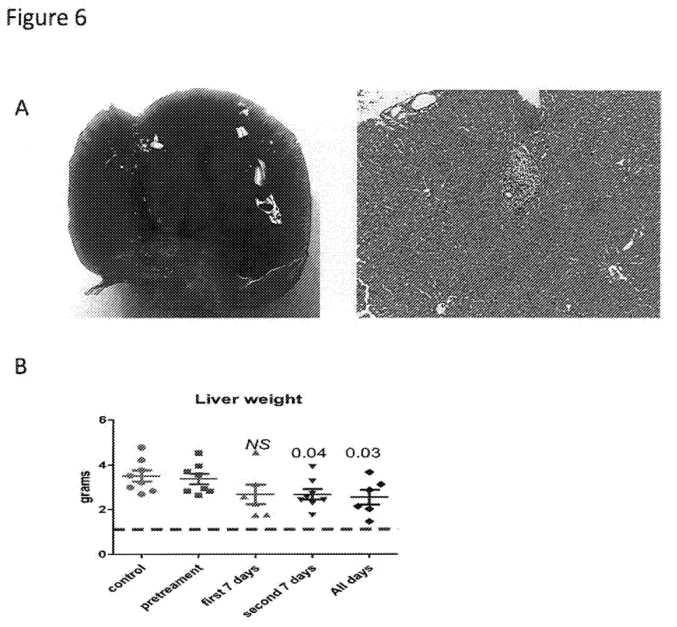

To determine if the anti-metastatic effects of CDK8/19 inhibition observed in the splenic injection model were due to the prevention of the initial establishment of hepatic metastases or growth inhibition of already-established metastases, we asked whether Senexin B can inhibit metastatic growth in the liver when the drug is administered after the metastases have been established. In agreement with previous characterization of the time course of hepatic metastasis following splenic injection of CT26 cells (Vidal-Vanaclocha, 2008), we found macroscopically and microscopically detectable metastatic tumors in the livers of mice sacrificed 7 days after splenic inoculation (FIG. 6A), indicating that the effect of a drug administered at a later point would have to involve suppression of metastatic growth. We compared the effects of Senexin B dimaleate on hepatic metastasis, when the drug was administered by gavage at 50 mg/kg doses in CD vehicle, b.i.d. two days prior to splenic inoculation, during the first week after inoculation, during the second week after inoculation, or over the entire two-week period following splenic inoculation of CT26 cells. The weights of livers collected two weeks after inoculation (FIG. 6B) demonstrate that drug administration prior to inoculation had no effect on liver metastasis. Drug administration during the first week decreased the liver weights but this effect did not reach statistical significance. On the other hand, treatments administered during both weeks or only for the second week after inoculation were equally efficient in suppressing metastatic growth in the liver, indicating that CDK8/19 inhibition inhibits the growth of already-established liver metastases (FIG. 6B).

### Example 3. CDKS/19 Inhibition Extends the Survival of Colon Cancer Liver Metastasis

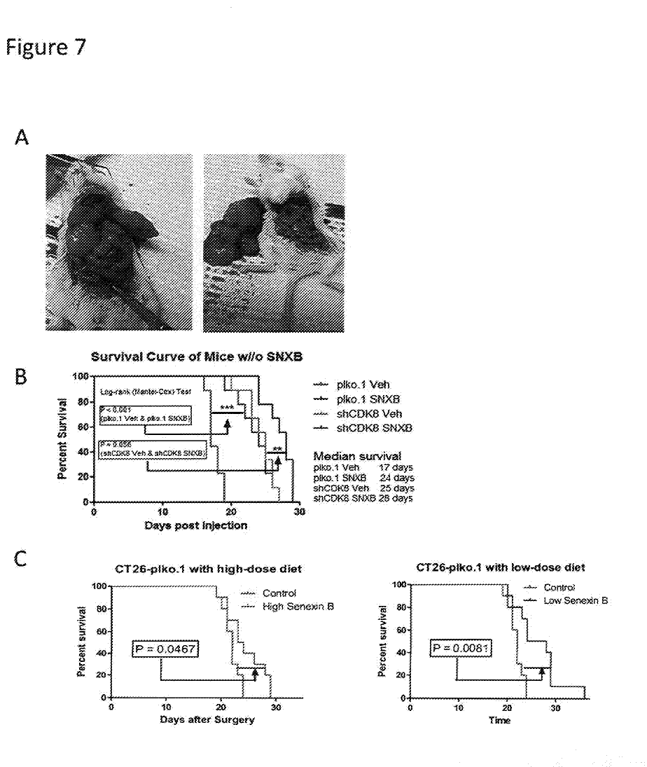

We have analyzed the survival of mice after splenic injection of 2×10⁵ CT26 cells into 8 week old female BALB/c mice, followed by removal of the spleen 1 minute after injection. Mice were sacrificed when paralysis, lack of movement or paleness (due to abdominal hemorrhage) occurred. The sacrificed mice showed extensive liver metastasis (FIG. 7A). Kaplan-Meyer (KM) survival plots in FIG. 7B shows that mice injected with shCDK8-1 cells showed significantly extended survival relative to mice inoculated with vector control cells. On the other hand, Senexin B dimaleate treatment by gavage at 50 mg/kg in CD vehicle, b.i.d. prolonged the survival of mice inoculated with vector control cells to a similar degree as the survival time of mice inoculated with shCDK8-1 cells (FIG. 7B). The combination of knockdown of CDK8 and treatment with Senexin B together increased the survival time even further (FIG. 7B). To establish serum drug concentrations associated with therapeutic efficacy in this model, we conducted a similar survival study where Senexin B dimaleate was administered by mixing the drug into mouse food, which provides for sustained drug delivery over time. Senexin B was mixed into food at two concentrations differing approximately 4-fold and designated low-dose and high-dose. Serum samples were collected from treated mice and Senexin B in the serum was measured by a LC/MS/MS assay. FIG. 7C shows KM survival plots for mice receiving the control diet (Research Diets, D12450B), the low-dose diet and the high-dose diet, along with the measurements of Senexin B serum concentrations in mice receiving the low- and the high-dose diets. There was no apparent toxicity and no significant change in mouse body weight in mice receiving Senexin B relative to control diet, as measured on days 8 and 15 after surgery. Remarkably, the low-dose diet, with an average serum concentration of just 58 nM, was at least as efficient as the high-dose diet, with an average serum concentration of 206 nM, indicating that hepatic colon cancer metastasis is very sensitive to CDK8/19 inhibition.

### Example 4. CDKS/19 Inhibitor Suppresses Hepatic Metastasis of Human Colon Cancer

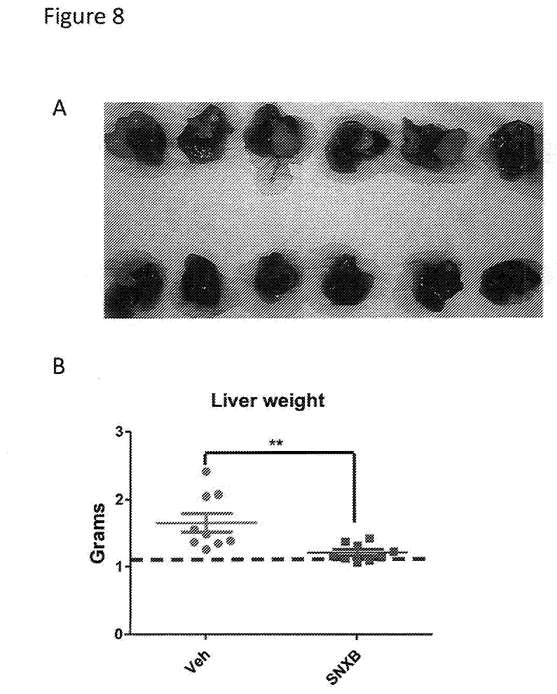

The splenic injection model was used to test the effect of Senexin B on hepatic metastasis of human HCTl 16 colon cancer cells, which are insensitive to small-molecule CDK8/19 inhibitors (Koehler et al., 2016; Pelish et al., 2015; Porter et al., 2012). 1×10⁶ HCTl 16 cells were injected into the spleens of athymic nude (nu/nu) mice (JAX #002019) mice (female, 8 weeks old), and spleens were removed 1 min later. Mice were treated with Senexin B dimaleate (50 mg/kg by gavage in CD vehicle, b.i.d.) or vehicle control (n=10). 7 weeks after tumor cell inoculation, mice were sacrificed and livers with tumor metastases (FIG. 8A) were collected. Liver weights showed strong inhibition of hepatic tumor growth by Senexin B in this otherwise insensitive cell line (FIG. 8B).

The results presented in Examples 1˜4 demonstrate that CDK8/19 inhibitors suppress the growth of colon cancer liver metastases even in those tumors that show little or no sensitivity to such inhibitors in the primary tumor setting. Based on these surprising results, CDK8/19 inhibitors can be used for the treatment of hepatic metastases in colon cancer patients.

### REFERENCE LIST

Adler, A. S., McCleland, M. L., Truong, T., Lau, S., Modrusan, Z., Soukup, T. M., Roose-Girma, M., Blackwood, E. M., and Firestein, R. (2012). CDK8 maintains tumor dedifferentiation and embryonic stem cell pluripotency. Cancer Res. 72, 2129-2139.
Alarcon, C., Zaromytidou, A. I., Xi, Q., Gao, S., Yu, J., Fujisawa, S., B arlas, A., Miller, A. N., Manova-Todorova, K., Macias, M. J., Sapkota, G., Pan, D., and Massague, J. (2009). Nuclear CDKs drive Smad transcriptional activation and turnover in BMP and TGF-beta pathways. Cell 139, 757-769.
Bragelmann, J, Klumper, N., Offermann, A, von, M. A., Bohm, D, Deng, M., Queisser, A., Sanders, C., Syring, !., Merseburger, A. S., Vogel, W., Sievers, E., Vlasic, I., Carlsson, J., Andren, O., Brossart, P., Duensing, S., Svensson, M. A., Shaikhibrahim, Z., Kirfel, J., and Pemer, S. (2016). Pan-Cancer Analysis of the Mediator Complex Transcriptome Identifies CDK19 and CDK8 as Therapeutic Targets in Advanced Prostate Cancer. Clin. Cancer Res. DOI: 10.1158/1078-0432.CCR-16-0094.
Broude E V, Gyorffy, B., Chumanevich, AP., Chen, M., McDermott, M. S., Shtutman, M., Catroppo, James F., and Roninson L B. (2015) Expression of CDK8 and CDK8-interacting genes as potential biomarkers in breast cancer. Curr. Cancer Drug Targets. 15, 739-749.
Cee, V. J., Chen, D. Y., Lee, M. R., and Nicolaou, K. C. (2009). Cortistatin A is a high-affinity ligand of protein kinases ROCK, CDK8, and CDKI 1. Angew. Chem. Int. Ed Engl. 48, 8952-8957.
Clarke, P. A., Ortiz-Ruiz, M. J., TePoele, R., Adeniji-Popoola, O., Box, G., Court, W., Czasch, S., E1, B. S., Esdar, C., Ewan, K., Gowan, S., de Haven, B. A., Hewitt, P., Hobbs, S. M., Kaufinann, W., Mallinger, A., Raynaud, F., Roe, T., Rohdich, F., Schiemann, K, Simon, S., Schneider, R.,
Valenti, M., Weigt, S., Blagg, J., Blaukat, A., Dale, T. C., Eccles, S. A., Hecht, S., Urbahns, K., Workman, P., and Wienke, D. (2016). Assessing the mechanism and therapeutic potential of modulators of the human Mediator complex-associated protein kinases. Elife. 5. DOI: http://dx.doi.org/10.7554/eLife.20722
Czodrowski, P., Mallinger, A., Wienke, D., Esdar, C., Poeschke, O., Busch, M., Rohdich, F., Eccles, S. A., Ortiz Ruiz, M. J., Schneider, R., Raynaud, F. I., Clarke, P. A., Musil, D., Schwarz, D., Dale, T. C., Urbahns, K., Blagg, J., and Schiemann, K. (2016). Structure-based optimization of potent, selective and orally bioavailable CDK8 inhibitors discovered by high throughput screening. J. Med. Chem. 59, 9337-9349.
Donner, A. J., Ebmeier, C. C., Taatjes, D. J., and Espinosa, J. M. (2010). CDK8 is a positive regulator of transcriptional elongation within the serum response network. Nat. Struct. Mol. Biol. 17, 194-201.
Firestein, R., Bass, A. J., Kim, S. Y., Dunn, I. F., Silver, S. J., Guney, I., Freed, E., Ligon, A. H., Vena, N., Ogino, S., Chheda, M. G., Tamayo, P., Finn, S., Shrestha, Y., Boehm, J. S., Jain, S., Bojarski, E., Mermel, C., Barretina, J., Chan, J. A., Baselga, J., Tabemero, J., Root, D. E., Fuchs, C. S., Loda, M., Shivdasani, R. A., Meyerson, M., and Hahn, W. C. (2008). CDK8 is a colorectal cancer oncogene that regulates beta-catenin activity. Nature 455, 547-551.
Fryer, C. J., White, J. B., and Jones, K. A. (2004). Mastermind recruits CycC:CDK8 to phosphorylate the Notch ICD and coordinate activation with turnover. Mol. Cell 16, 509-520. Galbraith, M. D., Allen, M. A., Bensard, C. L., Wang, X., Schwinn, M. K., Qin, B., Long, H. W., Daniels, D. L., Hahn, W. C., Dowell, R. D., and Espinosa, J. M. (2013). HIF1A employs CDK8-mediator to stimulate RNAPII elongation in response to hypoxia. Cell 153, 1327-1339.
Galbraith, M. D., Donner, A. J., and Espinosa, J. M. (2010). CDK8: a positive regulator of transcription. Transcription. 1, 4-12.
Griswold, D. P. and Corbett, T. H. (1975). A colon tumor model for anticancer agent evaluation. Cancer 36, 2441-2444.
Kapoor, A., Goldberg, M. S., Cumberland, L. K., Ratnakumar, K., Segura, M. F., Emanuel, P. O., Menendez, S., Vardabasso, C., Leroy, G., Vidal, C. I., Polsky, D., Osman, !., Garcia, B. A., Hemando, E., and Bemstein, E. (2010). The histone variant macroH2A suppresses melanoma progression through regulation of CDK8. Nature 468, 1105-1109.
Koehler, M. F., Bergeron, P., Blackwood, E. M., Bowman, K., Clark, K. R., Firestein, R.,
Kiefer, J. R., Maskos, K., McCleland, M. L., Orren, L., Salphati, L., Schmidt, S., Schneider, E. V., Wu, J., and Beresini, M. H. (2016). Development of a Potent, Specific CDK8 Kinase Inhibitor Which Phenocopies CDK8/19 Knockout Cells. ACS Med. Chem. Lett. 7, 223-228.
Lafreniere, R. and Rosenberg, S. A. (1986). A novel approach to the generation and identification of experimental hepatic metastases in a murine model. J. Natl. Cancer Inst. 76, 309-322.
Mallinger, A., Schiemann, K., Rink, C., Stieber, F., Calderini, M., Crumpler, S., Stubbs, M., Adeniji-Popoola, O., Poeschke, O., Busch, M., Czodrowski, P., Musil, D., Schwarz, D., Ortiz-Ruiz, M. J., Schneider, R., Thai, C., Valenti, M., de Haven, B. A., Burke, R., Workman, P., Dale, T., Wienke, D., Clarke, P. A., Esdar, C., Raynaud, F. I., Eccles, S. A., Rohdich, F., and Blagg, J. (2016). Discovery of Potent, Selective, and Orally Bioavailable Small-Molecule Modulators of the Mediator
Complex-Associated Kinases CDK8 and CDK19. J. Med. Chem. 59, 1078-1101.
Manfredi, S., Lepage, C., Hatem, C., Coatmeur, O., Faivre, J., and Bouvier, A. M. (2006).
Epidemiology and management of liver metastases from colorectal cancer. Ann. Surg. 244, 254-259.
McCleland, M. L., Soukup, T. M., Liu, S. D., Esensten, J. H., de Sousa E Melo, Yaylaoglu, M., Warming, S., Roose-Girma, M., and Firestein, R. (2015). Cdk8 deletion in the Apc(Min) murine tumour model represses EZH2 activity and accelerates tumourigenesis. J. Pathol. 237, 508-519. McDermott, M. S., Chumanevich, A. A., Lim, C. U., Liang, J., Chen, M., Altilia, S., Oliver, D., Rae, J. M., Shtutman, M., Kiaris, H., Gyorffy, B., Roninson, I. B., and Broude, E. V. (2017).
Inhibition of CDK8 mediator kinase suppresses estrogen dependent transcription and the growth of estrogen receptor positive breast cancer. Oncotarget. doi: 10.18632/oncotarget.14894.
Pelish, H. E., Liau, B. B., Nitulescu, 1.1., Tangpeerachaikul, A., Poss, Z. C., Da Silva, D. H., Caruso, B. T., Arefolov, A., Fadeyi, O., Christie, A. L., Du, K., Banka, D., Schneider, E. V., Jestel, A.,
Zou, G., Si, C., Ebmeier, C. C., Bronson, R. T., Krivtsov, A. V., Myers, A. G., Kohl, N. E., Kung, A. L., Armstrong, S. A., Lemieux, M. E., Taatjes, D. J., and Shair, M. D. (2015). Mediator kinase inhibition further activates super-enhancer-associated genes in AML. Nature 526, 273-276.
Porter, D. C., Farmaki, E., Altilia, S., Schools, G. P., West, D. K., Chen, M., Chang, B. D., Puzyrev, A. T., Lim, C. U., Rokow-Kittell, R., Friedhoff, L. T., Papavassiliou, A. G., Kalurupalle, S., Hurteau, G., Shi, J., Baran, P. S., Gyorffy, B., Wentland, M. P., Broude, E. V., Kiaris, H., and Roninson, I. B. (2012). Cyclin-dependent kinase 8 mediates chemotherapy-induced tumor-promoting paracrine activities. Proc. Natl. Acad. Sci. U. S. A 109, 13799-13804.
Porter, D. C., Chen, M., Liang, J., Kaza, V., Chumanevich, A, Altilia, S., Farmaki, E., Pena, M., Schools, G. P., Chatzistamou, I., Friedhoff, L. T., Wentland, M. P., Broude, E. V., Kiaris, H. and Roninson, I. B. Abstract PRO8: Targeting tumor microenvironment with selective small-molecule inhibitors ofCDK8/19. DOI: 10.1158/1538-7445.CHTME14-PRO8 Published 1 Jan. 2015. http://cancerres.aacrjournals.org/content/75/1_Supplement/PRO8
Porter, D. C., Liang, J., Kaza, V., Chumanevich, A. A, Altilia, S., Farmaki, E., Chen, M., Schools, G. P., Chatzistamou, I., Pena, M. M., Friedhoff, L. T., Wentland, M. P., Broude, E., Kiaris, H., and Roninson, I. B. Abstract 4879: Targeting the seed and the soil of cancers with selective small-molecule inhibitors of CDK8/19: Chemopotentiating, chemopreventive, anti-invasive and anti-metastatic activities. DOI: 10. 11 58/1538-7445.AM2014-4879 Published 1 Oct. 2014. http://cancerres.aacrjournals.org/content/74/19_Supplement/4879
Putz, E. M., Gotthardt, D., Hoermann, G., Csiszar, A., Wirth, S., Berger, A., Straka, E., Rigler, D., Wallner, B., Jamieson, A. M., Pickl, W. F., Zebedin-Brandl, E. M., Muller, M., Decker, T., and Sexl, V. (2013). CDK8-mediated STAT1-S727 phosphorylation restrains NK cell cytotoxicity and tumor surveillance. Cell Rep. 4, 437-444.
Rzymski, T., Mikula, M., Wiklik, K., and Brzozka, K. (2015). CDK8 kinase—An emerging target in targeted cancer therapy. Biochim. Biophys. Acta.
Tsutsui, T., Fukasawa, R., Tanaka, A., Hirose, Y., and Ohkuma, Y. (2011). Identification of target genes for the CDK subunits of the Mediator complex. Genes Cells 16, 1208-1218. Vidal-Vanaclocha, F. (2008). The prometastatic microenvironment of the liver. Cancer Microenviron. 1, 113-129.
Westerling, T., Kuuluvainen, E., and Makela, T. P. (2007). Cdk8 is essential for preimplantation mouse development. Mol. Cell Biol. 27, 6177-6182.
Xu, D., Li, C. F., Zhang, X., Gong, Z., Chan, C. H., Lee, S. W., Jin, G., Rezaeian, A. H., Han, F., Wang, J., Yang, W. L., Feng, Z. Z., Chen, W., Wu, C. Y., Wang, Y. J., Chow, L. P., Zhu, X. F., Zeng, Y. X., and Lin, H. K. (2015a). Skp2-MacroH2A1-CDK8 axis orchestrates G2/M transition and tumorigenesis. Nat. Commun. 6, 6641.
Xu, W., Wang, Z., Zhang, W., Qian, K., Li, H., Kong, D., Li, Y., and Tang, Y. (2015b). Mutated K-ras activates CDK8 to stimulate the epithelial-to-mesenchymal transition in pancreatic cancer in part via the Wnt/beta-catenin signaling pathway. Cancer Lett. 356, 613-627.
Zhang, B., Halder, S. K., Zhang, S., and Datta, P. K. (2009). Targeting transforming growth factor-beta signaling in liver metastasis of colon cancer. Cancer Lett. 277, 114-120.
Zylkiewicz, E., et al. “402-SEL120-34A, a specific, potent and orally bioavailable inhibitor of CDK8, targets STAT-dependent gene transcription in leukemia and lymphoma models.” European Journal of Cancer 69 (2016): S132.

What is claimed is:

1. A method for treating hepatic metastatic colon cancer in a subject, the method comprising administering to the subject a small molecule selective inhibitor of CDK8/19 at a dosage that inhibits growth of the hepatic metastatic colon cancer, and does not cause a dose-limiting toxicity.

2. A method for treating a subject having both a primary colon cancer tumor and hepatic metastatic colon cancer, the method comprising administering to the subject a small molecule selective inhibitor of CDK8/19 at a dosage that inhibits growth of the hepatic metastatic colon cancer, but does not significantly inhibit growth of the primary colon cancer tumor.

3. The method according to claim 2, further comprising treating the primary colon cancer tumor.

4. The method according to claim 3, wherein treatment of the primary colon cancer tumor comprises surgery.

5. The method according to claim 3, wherein treatment of the primary colon cancer tumor comprises radiation therapy.

6. The method according to claim 3, wherein treatment of the primary colon cancer tumor comprises chemotherapy.


SEQUENCE LISTING (6 sequences)

SEQ ID NO: 1 - DNA, 19 bp, Artificial Sequence
  Synthetic- PCR primer for the detection of CDK8
cgggtcgaggacctgtttg

SEQ ID NO: 2 - DNA, 23 bp, Artificial Sequence
  Synthetic- PCR primer for the detection of CDK8
tgccgacatagaaattccagttc

SEQ ID NO: 3 - DNA, 21 bp, Artificial Sequence
  Synthetic- PCR primer for the detection of CDK19
ggtcaagcctgacagcaaagt

SEQ ID NO: 4 - DNA, 21 bp, Artificial Sequence
  Synthetic- PCR primer for the detection of CDK19
ttcctggaagtaagggtcctg

SEQ ID NO: 5 - DNA, 21 bp, Artificial Sequence
  Synthetic- shRNA targeting CDK8 (shCDK8-l)
ctaacgtcagaaccaatattt

SEQ ID NO: 6 - DNA, 21 bp, Artificial Sequence
  Synthetic- shRNA targeting CDK8 (shCDK8-2)
gtcttatcagtgggttgattc